(19) United States
(12) Patent Application Publication
(10) Pub. No.: US 2026/0092024 A1
(43) Pub. Date: Apr. 2, 2026

(54) METHODS FOR DEHYDROGENATING HYDROCARBONS

(71) Applicant: Dow Global Technologies LLC, Midland, MI (US)

(72) Inventors: Anthony Plauck, Sugarland, TX (US); Charmaine Bennett, Houston, TX (US); Kevin Blann, Missouri City, TX (US); Barry B. Fish, Lake Jackson, TX (US); Jordan Keith, Pearland, TX (US); Davy Nieskens, Terneuzen (NL); Matthew T. Pretz, Lake Jackson, TX (US)

(73) Assignee: Dow Global Technologies LLC, Midland, MI (US)

(21) Appl. No.: 19/111,463

(22) PCT Filed: Sep. 12, 2023
(86) PCT No.: PCT/US2023/073962

§ 371 Date: Mar. 13, 2025

Related U.S. Application Data
(60) Provisional application No. 63/406,440, filed on Sep. 14, 2022.

(51) Int. Cl. C07C 5/48

(52) U.S. Cl. CPC C07C 5/48

(57) ABSTRACT

According to one or more embodiments described herein, a method for dehydrogenating hydrocarbons may include contacting a feed stream including one or more hydrocarbons with a particulate solid, which may include an oxygen-carrier material, to form hydrogen and one or more products. At least a portion of the hydrogen may be reacted with oxygen from the oxygen-carrier material to form water and reduce the oxygen content in the oxygen-carrier material. The process may include passing the particulate solid from the dehydrogenation reactor to a processing zone. In the processing zone, a fuel may be injected into the processing zone, and at least a portion of the fuel may react with oxygen from the oxygen-carrier material of the particulate solid. The gases in the processing zone may include less than or equal to 1 mol. % O₂.

### CROSS REFERENCE TO RELATED APPLICATION

This application claims priority to U.S. Provisional Application Ser. No. 63/406,440, filed Sep. 14, 2022, which is hereby incorporated by reference in its entirety.

### TECHNICAL FIELD

Embodiments described herein generally relate to chemical processing and, more specifically, to processes and systems utilized for dehydrogenation of chemical species.

### BACKGROUND

Olefinic compounds may be utilized as base materials to produce many types of goods and materials. For example, ethylene may be utilized to manufacture polyethylene, ethylene chloride, or ethylene oxides. Such products may be utilized in product packaging, construction, textiles, etc. Thus, there is an industry demand for olefinic compounds, such as ethylene, propylene, butene, and styrene.

### SUMMARY

One method for producing olefinic compounds is by dehydrogenating hydrocarbons. The dehydrogenation reaction may be promoted by reducing or removing hydrogen formed during dehydrogenation by reacting the hydrogen with oxygen to form water, which pushes the equilibrium towards the olefinic compound products. In such embodiments, an oxygen-carrier material may be utilized to provide the oxygen that reacts with the hydrogen. The oxygen-carrier material may be included in a particulate solid. Such oxygen-carrier materials may cycle through the dehydrogenation reactor to a processing zone, and then a regeneration unit.

In some systems, fuels may be utilized to heat the particulate solids, which may subsequently supply heat for the dehydrogenation reaction. In conventional embodiments, this fuel may be combusted in the regeneration unit. However, it has been discovered that combusting the fuel in the processing zone may have beneficial results. In such embodiments, the fuel may combust with oxygen from the oxygen-carrier material in the processing zone, such that little or no oxygen gas is supplied to the processing zone, which may diminish stripping performance. Additionally, in some embodiments hydrogen is utilized as the fuel and, thus, water is formed in the processing zone, which is subsequently combined with the products of the dehydrogenation reaction, and can be subsequently separated from the products with relative ease.

According to one or more embodiments described herein, hydrocarbons may be dehydrogenated by a process comprising contacting a feed stream comprising one or more hydrocarbons with a particulate solid in a dehydrogenation reactor. The particulate solid may comprise an oxygen-carrier material. In the dehydrogenation reactor one or more hydrocarbons may be dehydrogenated to form hydrogen and one or more products. At least a portion of the hydrogen may be reacted with oxygen from the oxygen-carrier material to form water and reduce the oxygen content in the oxygen-carrier material. The process may further comprise passing the particulate solid from the dehydrogenation reactor to a processing zone. A fuel may be injected into the processing zone. In the processing zone, at least a portion of the fuel may react with oxygen from the oxygen-carrier material of the particulate solid. The gases in the processing zone may comprise less than or equal to 1 mol. % O₂. The process may further comprise passing at least a portion of the particulate solid from the processing zone to a regeneration unit. The particulate solid may be exposed to an oxygen-containing gas in the regeneration unit, such that the content of oxygen in the oxygen-carrier material of the particulate solid may be increased in the regeneration unit. The process may further comprise passing at least a portion of the particulate solid from the regeneration unit to the dehydrogenation reactor.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawings and claims, or recognized by practicing the described embodiments. The drawings are included to provide a further understanding of the embodiments and, together with the detailed description, serves to explain the principles and operations of the claimed subject matter. However, the embodiments depicted in the drawings are illustrative and exemplary in nature, and not intended to limit the claimed subject matter.

### BRIEF DESCRIPTION OF THE DRAWINGS

Reference will now be made in greater detail to various embodiments, some of which are illustrated in the accompanying drawings, wherein:

FIG. 1 schematically depicts a reactor system, according to one or more embodiments of the present disclosure;

FIG. 2 schematically depicts a reactor system according to alternative embodiments of the present disclosure.

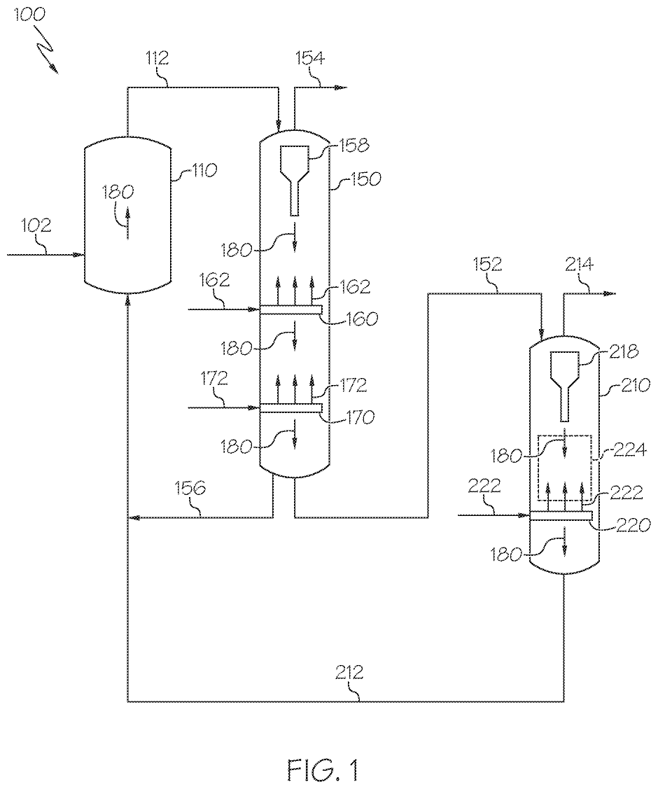

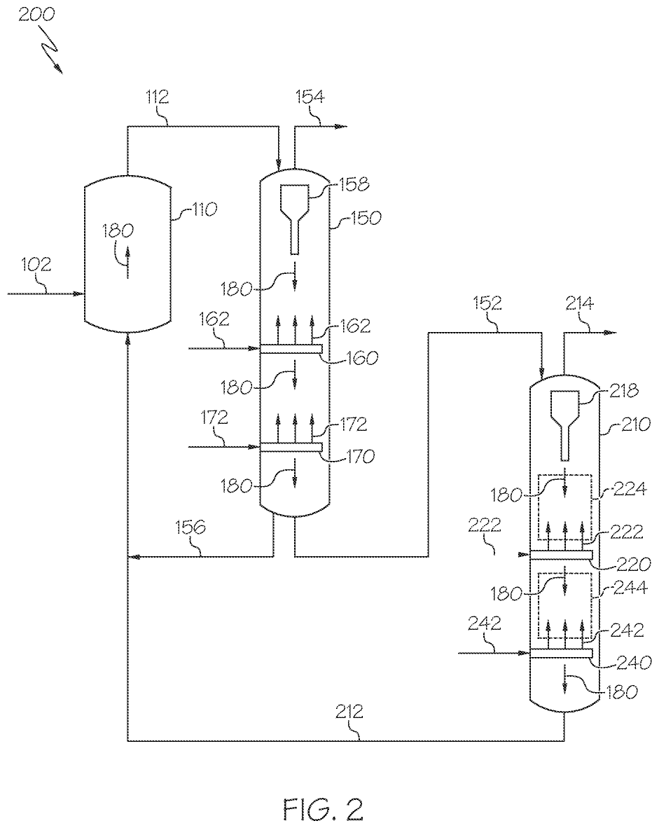

When describing the simplified schematic illustration of FIG. 1 and FIG. 2, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

### DETAILED DESCRIPTION

Specific embodiments of the present application will now be described. The disclosure may, however, be embodied in different forms and should not be construed as limited to the embodiments set forth in this disclosure. Rather, these embodiments are provided so that this disclosure will be thorough and complete, and will fully convey the scope of the subject matter to those skilled in the art.

Embodiments of the methods presently disclosed will now be described herein in detail in the context of the reactor systems of FIG. 1 and FIG. 2 operating as circulating fluidized bed processes to dehydrogenate hydrocarbons. However, it should be understood that the principles disclosed and taught herein may be applicable to other systems which utilize different system components oriented in different ways. For example, the concepts described herein may be equally applied to other systems with alternate reactor units and regeneration units, such as those that operate under non-fluidized conditions or include downers rather than risers, and vice versa. It should be further understood that not all portions of the reactor systems of FIG. 1 and FIG. 2 should be construed as essential to the claimed subject matter. Moreover, while the recited method steps in the appended claims are described herein in the context of the reactor systems of FIG. 1 and FIG. 2, such recited method steps should be understood as adaptable to other systems, as would be understood by those skilled in the art.

Now referring to FIG. 1, an example reactor system 100 that may be suitable for use with the methods described herein is schematically depicted. The reactor system 100 may include a dehydrogenation reactor 110, a processing zone 150, and a regeneration unit 210. A feed stream 102 may be passed into the dehydrogenation reactor 110. A particulate solid 180 may be passed into the dehydrogenation reactor 110 via stream 212. The particulate solid 180 may be contacted with the feed stream 102 in the dehydrogenation reactor 110. The particulate solid 180 may comprise an oxygen-carrier material. The feed stream 102 may comprise one or more hydrocarbons which may be dehydrogenated in the dehydrogenation reactor 110 to form one or more products and hydrogen. Oxygen from the oxygen-carrier material may react with the hydrogen to form water. The one or more products and the water may exit the dehydrogenation reactor 110 via stream 112.

The particulate solid 180 may also be passed from the dehydrogenation reactor 110 to the processing zone 150 via stream 112. The processing zone 150 may comprise a gas/solids separator 158. The particulate solid 180 may be separated from the gases in stream 112 by the separator 158. The processing zone 150 may comprise a first gas inlet 160 and a second gas inlet 170. A fuel 162 may enter the processing zone 150 through the first gas inlet 160 and optionally a stripping gas 172 may enter the processing zone 150 through the second gas inlet 170. The particulate solid 180 may travel in a generally downwards direction through the processing zone 150. The gases within the processing zone 150 such as the fuel 162 may travel in a generally upwards direction through the processing zone 150 such that the particulate solid 180 and the gases move in a generally countercurrent flow pattern through the processing zone 150. The particulate solid 180 may then exit from the processing zone 150 via stream 152 and may be passed to a regeneration unit 210.

The regeneration unit 210 may comprise a gas/solids separator 218 and a third gas inlet 220. An oxygen-containing gas 222 may enter into the regeneration unit 210 through the third gas inlet 220. The particulate solid 180 may travel in a generally downwards direction through the regeneration unit 210 first through the separator 218, then past the third gas inlet 220. The particulate solid 180 may then exit from the regeneration unit 210 and be passed back to the dehydrogenation reactor 110 via stream 212.

In some embodiments, reactor system 100 may be operable to perform a circulating fluidized bed (CFB) dehydrogenation process. The CFB dehydrogenation process may include a dehydrogenation reactor 110, a processing zone 150, and a regeneration unit 210 all fluid bed based.

As discussed previously, in one or more embodiments, the feed stream 102 may be passed into the dehydrogenation reactor 110. In one or more embodiments, the feed stream 102 may comprise one or more hydrocarbons. In one or more embodiments, the one or more hydrocarbons may comprise an alkyl moiety. As used in the present disclosure a hydrocarbon comprises an “alkyl moiety” if the molecule has at least one carbon-carbon single bond capable of being dehydrogenated to form a carbon-carbon double bond. In one or more embodiments, the one or more hydrocarbons may comprise one or more of ethane, propane, butane, or ethylbenzene. According to one or more embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of ethane. In additional embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of propane. In additional embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of butane. In additional embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of ethylbenzene. In additional embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of the sum of ethane, propane, butane and ethylbenzene.

According to additional embodiments, the dehydrogenation reactor 110 may operate with a “back-mixed” fashion where the feed stream 102 enters the reactor, as to closely approximate isothermal conditions. As such, the fluid velocity at this region may be low enough and the particulate solid 180 flux may be great enough such that a dense bed may form at or around where the feed stream 102 is injected. In some embodiments, the superficial velocity of the reactor may be from 3-80 ft/s, such as from 3-40 ft/s, or 10-30 ft/s. The particulate solid 180 flux in the reactor may be from 1-300 lb/ft²-s, such as from 40-200 lb/ft²-s, or from 60-160 lb/ft²-s. The reactor may include multiple diameters, and may include one or more frustums to increase or decrease particulate solid 180 and/or gaseous reactant velocity. The reactor may operate with a gas residence time of from 0.1-10 seconds, such as from 0.5-6 seconds.

A particulate solid 180 may be utilized in the general operation of the reactor system 100. As used herein, the term “particulate solid” may refer to one or more solid particles suitable for fluidization. In one or more embodiments, the particulate solid 180 may comprise an oxygen-carrier material and a dehydrogenation catalyst material. In some embodiments, the particulate solid 180 may consist essentially of the oxygen-carrier material. As described herein, “consists essentially of” refers to materials with less than 1 wt. % of the non-recited materials (i.e., consisting essentially of A means A is at least 99 wt. % of the composition). In some embodiments, the particulate solid 180 may not comprise a dehydrogenation catalyst material. In some embodiments, the oxygen-carrier material and the dehydrogenation catalyst material may be separate particles of the particulate solid 180. In some embodiments, the oxygen-carrier material and the dehydrogenation catalyst may be contained in the same particles of the particulate solid 180.

In embodiments where the particulate solid 180 comprises a dehydrogenation catalyst, the dehydrogenation of the one or more hydrocarbons may be at least partially by catalytic dehydrogenation. Catalytic dehydrogenation is the dehydrogenation of a hydrocarbon that is promoted by the use of a dehydrogenation catalyst. In embodiments, where the particulate solid 180 does not comprise a dehydrogenation catalyst the dehydrogenation of the one or more hydrocarbons may be by non-catalytic thermal dehydrogenation. Non-catalytic thermal dehydrogenation refers to the dehydrogenation of a hydrocarbon that occurs without the use of a dehydrogenation catalyst and instead may occur because of high temperature, pressure or combinations thereof.

In some embodiments, the particulate solid 180 may comprise a “dual-purpose material” that may act as both a dehydrogenation catalyst as well as an oxygen-carrier material. It should be understood that, in at least the embodiments described herein where an oxygen-carrier material and a dehydrogenation catalyst are utilized in the same reaction vessel (such as those of FIG. 1), such a dual-purpose material may be utilized either in replacement or in combination with the oxygen-carrier material of the particulate solid 180 or the dehydrogenation catalyst of the particulate solid 180.

Unless specified herein, an “oxygen-carrier material” may generally refer to an oxygen-rich oxygen-carrier material or an oxygen-deficient oxygen-carrier material. Unless specified herein, the “dual-purpose material” may generally refer to an oxygen-rich dual-purpose material or an oxygen-deficient dual-purpose material. For example, an oxygen-deficient state may be present after some oxygen is utilized for combustion and may be oxygen-rich prior to the combustion, following regeneration of the oxygen-deficient state material. The reactions may take place in one or more fluidized bed reactors, such as circulating fluidized bed reactors. The reactors may be, for example, risers or downers.

As described herein, in one or more embodiments, the dehydrogenation catalyst and the oxygen-carrier material may be separate particles of the particulate solid 180. One contemplated advantage of such a system is that by adding, removing, or substituting one or both of the dehydrogenation catalyst and oxygen-carrier material, the functionality of the system can be altered, even when the system is on-line. For example, the reaction heat load could be adjusted by adding or removing one or both of the dehydrogenation catalyst and the oxygen-carrier material. This may be advantageous, in some embodiments, as compared with a dual purpose material, since the dual purpose particle's heat balance must be determined prior to reaction and cannot be easily adjusted by varying the amount of dehydrogenation catalyst versus oxygen-carrier material. Control of the ratio of dehydrogenation catalyst versus oxygen-carrier material may further be advantageous since reaction selectivity may be better tuned. For example, the amount of hydrogen in the system may be used to control the degree of combustion, or component balances may be used to optimize downstream separation processes.

In some embodiments, the dehydrogenation reactor 110 may include from 1 wt. % to 100 wt. %, such as from 5 wt. % to 95 wt. %, or from 75 wt. % to 25 wt. %, oxygen-carrier material based on the total weight of the particulate solid 180 in the dehydrogenation reactor 110. In other embodiments, the dehydrogenation reactor 110 may include from 50 wt. % to 75 wt. % oxygen-carrier material based on the total weight of the particulate solid 180 in the dehydrogenation reactor 110. In some embodiments, relatively large amounts of oxygen-carrier material may be present (e.g., at least 80 wt. %, at least 85 wt. %, or even at least 90 wt. %), particularly if there is relatively slow release of oxygen by the oxygen-carrier materials as compared to the rate of dehydrogenation. In some embodiments, the dehydrogenation reactor 110 may include from 0 wt. % to 99 wt. %, such as from 5 wt. % to 95 wt. %, or from 25 wt. % to 75 wt. %, dehydrogenation catalyst based on the total weight of the particulate solid 180 in the dehydrogenation reactor 110. In other embodiments, the dehydrogenation reactor 110 may include from 25 wt. % to 50 wt. % dehydrogenation catalyst based on the total weight of the particulate solid 180 in the dehydrogenation reactor 110. In some embodiments, the dehydrogenation reactor 110 may include up to 95 wt. %, 99 wt. %, or even 100 wt. % of dual-purpose material of the total weight of the particulate solid 180 in the dehydrogenation reactor 110. For example, all or a far majority of the particulate solid 180 may be dual-purpose material when utilized. In embodiments, the particulate solid 180 material may encompass all solids in the system aside from coke.

As stated previously, within the dehydrogenation reactor 110, hydrogen may be contacted with the oxygen-rich oxygen-carrier material of the particulate solid 180. The oxygen-carrier material may include one or more metal oxides. According to one or more embodiments, the one or more metal oxides may be a redox-active metal oxide or a mixture of redox-active metal oxides. The redox-active metal oxide includes binary, ternary, or other mixed metal oxides capable of undergoing reduction in the presence of a reducing agent (for example, hydrogen) and oxidation in the presence of oxidizing agent (for example, oxygen or air). In some embodiments, the redox-active metal oxide may be a metal MOx, where M may be one or more metals of IUPAC group 6, 7, 8, 9, 10, 11, or 12 and “x” is the number of associated oxygen atoms in the structure. For example the redox-active metal oxide may be Mn₂O₃, Fe₂O₃, Co₃O₄, CuO, (LaSr)CoO₃, (LaSr)MnO₃, Mg₆MnO₈, MgMnO₃, MnO₂, Fe₃O₄, Mn₃O₄, Cu₂O, NiO, Ni₂O₃, CrO, Cr₂O₃, CrO₂, ZnO, or any combination of other IUPAC group 6-12 metal oxide. In some embodiments, the redox-active metal oxide may be cerium oxide. For example, the redox-active metal oxide may be CeO₂, Ce₂O₃, or any other mixed metal oxide containing cerium. In further embodiments, the oxygen carrier material may include lanthanum oxide, La₂O₃, in combination with other reducible metal oxides. In some embodiments, the redox-active metal oxide may be chosen from Mn₂O₃, Fe₂O₃, Co₃O₄, CuO, (LaSr)CoO₃,(LaSr)MnO₃, Mg₆MnO₃, MgMnO₃, MnO₂, Fe₃O4, Mn₃O4, and Cu₂O. In some embodiments, the oxygen-carrier material may be a solid. In specific embodiments, the oxygen-carrier material may be a crushed solid or powder. In other embodiments, the oxygen-carrier material may be formulate using a redox-active metal oxide and a binder and/or support material to produce a fluidizable material with the require physical properties, for example, particle size distribution, density, and attrition resistance. The binder and/or support material may include alumina, silica, titania, magnesia, zirconia, or combinations thereof.

In one or more embodiments, the oxygen-carrier material may include a hydrogen-selective oxygen-carrier material that may include a promoter or a combination of various promoters. The addition of a promoter(s) may lead to the formation of a core-shell morphology. The promoter(s) may include alkali or alkaline-earth metal oxides from IUPAC group 1 and 2 and/or compounds comprising alkali-transition metal oxides or alkaline-earth transition metal oxides. In some embodiments, alkali elements may include one or more of sodium, lithium, potassium, and cesium. In some embodiments, alkaline-earth elements may include one or more of calcium, magnesium, strontium, and barium. In some embodiments, transition metals may include one or more of tungsten and molybdenum. For example, the one or more alkali or alkaline-earth transition metal oxides may be Na₂WO₄, K₂MOO₄, Na₂MoO₄, K₂WO₄, Li₂WO₄, CsWO₄, Li₂MoO₄, CaWO₄, CaMoO₄, MgWO₄, MgMoO₄, SrWO₄, SrMoO₄, BaWO₄ and BaMoO₄. In some embodiments, the promoter may include one or more of alkali or alkaline-earth metal salts selected from Group 1 and 2 metal cations and a counterion. In some embodiments, alkali elements may include one or more of sodium, lithium, potassium, and cesium. In some embodiments, alkaline-earth elements may include one or more of calcium, magnesium, strontium, and barium. In some embodiments, the counterion may include carbonates, sulphates, sulphites, sulfides, phosphates, phosphites and borates. For example, the alkali or alkaline-earth metal salts may be Na₂CO₃, Na₂SO₄, Na₃PO₄, Li₂CO₃, Li₂SO₄, Li₃PO₄, K₂CO₃, K₂SO₄, K₃PO₄, Cs₂CO₃, Cs₂SO₄, Cs₃PO₄, CaCO₃, CaSO₄, Ca₃(PO₄)₂, SrCO₃, SrSO₄, Sr₃(PO₄)₂, MgCO₃, MgSO₄, Mg₃(PO₄)₂, BaCO₃, BaSO₄, Ba₃(PO₄)₂, Na₂HPO₄, KHSO₄, Na₂SO₃, K₂B₄O₇, Na₃BO₃, or combinations thereof.

For example, oxygen-carrier materials such as those disclosed in U.S. App. No. 62/725,504, entitled “METHODS OF PRODUCING HYDROGEN-SELECTIVE OXYGEN CARRIER MATERIALS,” filed on, Aug. 31, 2018, and U.S. App. No. 62/725,508, entitled “HYDROGEN-SELECTIVE OXYGEN CARRIER MATERIALS AND METHODS OF USE,” filed on, Aug. 31, 2018, are contemplated as suitable for the presently disclosed processes, and the teachings of these references are incorporated by reference herein. In one or more additional embodiments, the oxygen-carrier material may include those of U.S. Pat. No. 5,430,209, U.S. Pat. No. 7,122,495, and/or WO 2018/232133, each of which are incorporated by reference in their entireties.

The oxygen-rich oxygen-carrier material may be reducible by releasing oxygen that may be selective for combusting hydrogen. For example, the oxygen-carrier material may be selective for the combustion of hydrogen over hydrocarbons. In some embodiments, the oxygen-rich oxygen-carrier material comprises from about 1 wt. % to about 20 wt. % releasable oxygen based on total weight of the oxygen-rich oxygen-carrier material. In other embodiments, the oxygen-rich oxygen-carrier material comprises from about 1 wt. % to about 10 wt. %, from about 1 wt. % to about 5 wt. %, from about 5 wt. % to about 20 wt. %, or from about 5 wt. % to about 10 wt. % releasable oxygen. As described herein, “releasable oxygen” may refer to the oxygen that can be released through redox by the oxygen-carrier material. Other oxygen may be present in the oxygen-carrier material that is not releasable through redox. It should be understood that in some embodiments, the oxygen may be released from a surface of the oxygen-carrier material simultaneously with the combustion of hydrogen at the surface of the oxygen-carrier material.

As stated previously, the releasable oxygen of the oxygen-rich oxygen-carrier materials may be selective for combusting hydrogen over hydrocarbons. In some embodiments, at least about 60% of the releasable oxygen of the oxygen-carrier material is selective for hydrogen combustion. In other embodiments, at least about 55% of the releasable oxygen of the oxygen-carrier material is selective for hydrogen combustion.

In embodiments, when the hydrogen is contacted by the oxygen-rich oxygen-carrier material, some of the releasable oxygen is removed from the oxygen-rich oxygen-carrier material. In some embodiments, contacting the hydrogen with the oxygen-rich oxygen-carrier material removes from about 1 wt. % to 50 wt. % of the releasable oxygen from the oxygen-rich oxygen-carrier material. In other embodiments, contacting the hydrogen with the oxygen-rich oxygen-carrier material removes from about 10 wt. % to about 50 wt. %, from about 10 wt. % to about 25 wt. %, or from about 25 wt. % to about 50 wt. % of the releasable oxygen from the oxygen-rich oxygen-carrier material.

In further embodiments, when the hydrogen is contacted by the oxygen-rich oxygen-carrier material, the oxygen-rich oxygen-carrier material combusts greater than about 50% of the hydrogen. In other embodiments, when the hydrogen is contacted by the oxygen-rich oxygen-carrier material, the oxygen-rich oxygen-carrier material combusts about 50% to about 90%, or about 75% to about 90% of the hydrogen that is produced.

The contacting of the oxygen-rich oxygen-carrier material with the hydrogen may combust the hydrogen and form an oxygen-diminished oxygen-carrier material. To form the oxygen-diminished oxygen-carrier material, at least a portion of the oxygen-rich oxygen-carrier material may be reduced to a lower oxidation state. Once the oxygen-carrier material has been reduced to form the oxygen-diminished oxygen-carrier material, the oxygen-diminished oxygen-carrier material may be discharged from the dehydrogenation reactor 110 at a lower oxidation state.

As described herein, in one or more embodiments, the particulate solid 180 may comprise a dehydrogenation catalyst. In one or more embodiments, the dehydrogenation catalyst may include gallium, chromium, and/or platinum. As described herein, a gallium and/or platinum dehydrogenation catalyst comprises gallium, platinum, or both. The dehydrogenation catalyst may be carried by an alumina or alumina silica support, and may optionally comprise potassium. In one or more embodiments, the dehydrogenation catalysts may include catalysts disclosed in U.S. Pat. No. 8,669,406, which is incorporated herein by reference in its entirety, such as those including Ga, Cr, and/or Fe based catalysts. According to additional embodiments, Pt based catalysts may be utilized. In one or more embodiments, those catalysts disclosed in EP 0948475B1 and/or WO 2010/133565, which are each incorporated herein by reference in its entirety, may be utilized. Additional catalyst embodiments contemplated as suitable for use in the systems and methods described herein include those of U.S. Pat. No. 8,669,406, which is incorporated herein by reference in its entirety. Such catalysts may contain relatively low amounts of Cr, such as less than 6%, or approximately 1.5%. However, it should be understood that other suitable dehydrogenation catalysts may be utilized to perform the dehydrogenation reaction.

In one or more embodiments, the dehydrogenation catalyst may exhibit suitable stability when in the presence of steam. As is described herein, the combustion of hydrogen may form steam, which may be in direct contact with the dehydrogenation catalyst. It is contemplated that not all dehydrogenation catalysts are equally effective in steam environments. In one or more embodiments, dehydrogenation catalysts are utilized which maintain a substantial amount of their reactivity and/or selectivity for the dehydrogenation of light alkanes. For example, one or more of the dehydrogenation catalysts utilized in the presently disclosed systems and methods may not deteriorate in alkane conversion and/or selectivity for dehydrogenation more than 25%, more than 20%, more than 15%, more than 10%, more than 5%, or may even have improved alkane conversion and/or selectivity for dehydrogenation when in the presence of steam in amounts consistent with the operation of the presently disclosed systems. In some embodiments, the dehydrogenation catalyst may function with such conversion and/or selectivity when exposed to at least 10 mol. % water (such as from 10 mol. % to 50 mol. % water) for a period of up to, e.g., 120 seconds (the time which the catalyst may be exposed to such conditions, according to some embodiments of the presently disclosed system).

Suitable examples of dehydrogenation catalysts may be prepared such that it meets the Geldart A definition. In some embodiments, the dehydrogenation catalyst comprises gallium and platinum supported on alumina in the delta or theta phase, or in a mixture of delta plus theta phases, or theta plus alpha phases, or delta plus theta plus alpha phases, modified with silica, and having a surface area preferably less than about 100 square meters per gram (m²/g), as determined by the BET method. In other embodiments, the dehydrogenation catalyst comprises: from 0.1 to 34 wt. %, preferably 0.2 to 3.8 wt. %, gallium oxide (Ga₂O₃); from 1 to 300 parts per million (ppm), preferably 50 to 300 ppm, by weight platinum; from 0 to 5 wt. %, preferably 0.01 to 1 wt. %, of an alkaline and/or earth-alkaline such as potassium; from 0.08 to 3 wt. % silica; the balance to 100 wt. % being alumina.

In one or more embodiments, heat may be gained or lost through the dehydrogenation reaction, the re-oxidation of the oxygen-diminished oxygen carrier material, and the reduction of the oxygen-rich oxygen carrier material may create or use heat (i.e., be exothermic or endothermic). In one or more embodiments, the contacting of the hydrocarbon feed with the dehydrogenation catalyst may be endothermic and results in a dehydrogenation heat loss. In some embodiments, the contacting of the hydrogen with the oxygen-rich oxygen carrier material may be exothermic and results in a combustion heat gain. The re-oxidizing of the oxygen-diminished oxygen carrier material may be exothermic and results in an oxygenation heat gain. As such, by incorporating hydrogen combustion during catalytic dehydrogenation, in some embodiments, enough heat may be generated during the re-oxidation of the oxygen-diminished oxygen carrier material to act as a source of heat for the alkane to olefin reaction. As such, embodiments of the disclosed process may allow for higher alkane conversion while reducing or eliminating needs for fuel gas, as required for conventional cracking because the heat gained throughout the process by the re-oxidizing of the oxygen carrier material, the combustion of hydrogen, or both may produce the amount of heat required for the alkanes or alkyl aromatics to olefins reaction.

As presently described, the “dehydrogenation heat loss” refers to the amount of heat lost by the dehydrogenation of the feed alkanes, the “combustion heat gain” refers to the amount of heat created by the combustion of the hydrogen, and the “oxygenation heat gain” refers to the amount of heat created by the oxidation of the oxygen-diminished oxygen carrier material. In one or more embodiments, the combustion heat gain may contribute heat to the system that account for at least a portion of the dehydrogenation heat loss. In additional embodiments, supplemental fuel 162 may be combusted to heat one or more of the dehydrogenation catalyst or the oxygen carrier material. The supplemental fuel 162 may make up for any shortcoming in heat created by the combustion of the hydrogen or the oxygenation of the oxygen carrier material. However, it should be understood that in the embodiments disclosed, the amount of necessary supplemental fuel 162 may be substantially less than would be necessary in a system that did not incorporate an oxygen carrier material.

The production of olefinic compounds by conventional dehydrogenation processes (e.g., those that do not incorporate hydrogen combustion) may be relatively expensive due to the high heat loads needed for the endothermic dehydrogenation reaction and/or the downstream separation steps sometimes needed to separate the unreacted hydrocarbons and remove hydrogen that is produced in the dehydrogenation reaction. Regarding reduced heat input, catalytic dehydrogenation processes are generally endothermic and require heat. However, the exothermic combustion of hydrogen can somewhat counterbalance that heat input requirement. Additionally, the oxygen carrier material, once diminished in oxygen content following the combustion, may be regenerated to regain its oxygen, which may be exothermic. This exothermic regeneration step may further counterbalance the heat input requirement to maintain the dehydrogenation reaction. In some embodiments, the heat produced by the oxygen carrier regeneration and combustion reaction may completely cover the heat needed for the endothermic dehydrogenation reaction and other heat demands such as heating the feed gases (air, hydrocarbon, etc.) or balancing heat losses, or at least reduce any supplemental fuel 162 needs of the system. For additional generalized information on dehydrogenation reactions that incorporate hydrogen combustion, those skilled in the art are referred to, for example, US Patent Publication 2021/0292259 A1, the teachings of which are incorporated herein by reference in their entirety.

Referring still to FIG. 1, during operation of the dehydrogenation reactor 110 of the reactor system 100, the feed stream 102 may enter a riser within the dehydrogenation reactor 110, and the product stream may exit the reactor system 100 via stream 112. According to one or more embodiments, the reactor system 100 may be operated by feeding a chemical feed (e.g., in a feed stream such as feed stream 102) into the dehydrogenation reactor 110.

According to one or more embodiments, a particulate solid 180 may be fed into the dehydrogenation reactor 110 via stream 152. The particulate solid 180 may include an oxygen-rich oxygen-carrier material. The particulate solid 180 may also include a dehydrogenation catalyst. The feed stream 102 may contact the particulate solid 180 in the dehydrogenation reactor 110. Each of the feed stream 102 and the particulate solid 180 may flow upwardly into and through the dehydrogenation reactor 110 to produce one or more products, an oxygen-diminished oxygen-carrier material, and hydrogen. In the dehydrogenation reactor 110 the one or more hydrocarbons in the feed stream 102 may be dehydrogenated to form one or more products and hydrogen. Additionally, within the dehydrogenation reactor 110, the hydrogen may be contacted with the oxygen-rich oxygen-carrier material in the dehydrogenation reactor 110. The oxygen-rich oxygen-carrier material may be reducible. The contacting of the oxygen-rich oxygen-carrier material with the hydrogen may combust the hydrogen and reduce the oxygen-carrier material to form an oxygen-diminished oxygen-carrier material and water.

In one or more embodiments, the one or more products may exit the dehydrogenation reactor 110 via stream 112. The one or more products may travel with the particulate solid 180 to the processing zone 150. The processing zone 150, as referred to herein, may include any vessel or portion of a vessel where stripping occurs. Generally, in the processing zone 150, the entrained or adsorbed hydrocarbons may be removed from the particulate solid. The one or more products may be separated from the particulate solid 180 in the processing zone 150 by a gas/solids separator 158. The one or more products may mix with other gases from the processing zone 150 and exit the processing zone 150 via stream 154. Stream 154 may be further processed such as by one or more subsequent separation steps or further reacted. It is contemplated that stream 154 may be utilized as a feed for another reactor system or sold as a chemical product. In embodiments, the one or more products in stream 154 may be mixed with water produced from reacting the hydrogen produced during the dehydrogenation of the one or more hydrocarbons and oxygen from the oxygen-carrier material. In some embodiments, the water may be removed from stream 154 and the one or more products utilizing a condenser.

As described above, in some embodiments, stream 154 may comprise one or more products. In one or more embodiments, the one or more products may comprise one or more olefinic compounds. As used herein, the term “olefinic compounds” refers to hydrocarbons having one or more carbon-carbon double bonds apart from the formal double bonds in aromatic compounds. For example, ethylene and styrene are olefinic compounds, but ethylbenzene would not be an olefinic compound as the only double bonds present in ethylbenzene are formal double bonds present as part of the aromatic structure. In one or more embodiments, the one or more olefinic compounds may comprise one or more of ethylene, propylene, butylene, or styrene. In some embodiments, the stream 154 may comprise at least 50 wt. %, at least 60 wt. %, at least 70 wt. %, at least 80 wt. %, at least 90 wt. %, at least 95 wt. % or even at least 99 wt. % of ethylene. In additional embodiments, stream 154 may comprise at least 50 wt. %, at least 60 wt. %, at least 70 wt. %, at least 80 wt. %, at least 90 wt. %, at least 95 wt. % or even at least 99 wt. % of propylene. In additional embodiments, stream 154 may comprise at least 50 wt. %, at least 60 wt. %, at least 70 wt. %, at least 80 wt. %, at least 90 wt. %, at least 95 wt. % or even at least 99 wt. % of butylene. In additional embodiments, stream 154 may comprise at least 50 wt. %, at least 60 wt. %, at least 70 wt. %, at least 80 wt. %, at least 90 wt. %, at least 95 wt. % or even at least 99 wt. % of styrene. In additional embodiments, stream 154 may comprise at least 50 wt. %, at least 60 wt. %, at least 70 wt. %, at least 80 wt. %, at least 90 wt. %, at least 95 wt. % or even at least 99 wt. % of the sum of one or more of ethylene, propylene, butylene, and styrene.

In one or more embodiments, the dehydrogenation reactor 110 may operate with a residence time of the vapor in the fluidized bed reactor of less than 10 seconds (such less than 9 seconds, less than 8 seconds, less than 7 seconds, less than 6 seconds, less than 5 seconds, less than 4 seconds, or even less than 3 seconds.

In one or more embodiments, the dehydrogenation reactor 110 may operate at a temperature of greater than or equal to 550° C. and less than or equal to 800° C. In some embodiments, the temperature in the dehydrogenation reactor 110 may be from 625 ° C. or 650° C. to 770° C. In other embodiments, the temperature in the dehydrogenation reactor 110 may be from 700° C. to 750° C. Without being bound by any particular theory, it is believed that too low of temperature (e.g., 550° C. or less) may limit the maximum conversion of the hydrocarbon due to equilibrium constraints as well as lowers the rate of dehydrogenation by the thermal and catalytic component. Too low of temperatures may also result in a slow rate of oxygen release from the oxygen-carrier material and low hydrogen combustion. On the other hand, high temperatures (e.g., greater than 800° C.) may result in thermal degradation of the products produced and may result in a lower product selectivity than is economically feasible. In some embodiments, the primary feed component(s) may be propane, ethylbenzene, and/or butane, and the dehydrogenation reactor 110 may operate at a temperature of greater than 600° C. In additional embodiments, the primary feed component may be ethane, and the dehydrogenation reactor 110 may operate at a temperature of at least 625 ° C.

In some embodiments, the dehydrogenation reactor 110 may operate at a pressure of at least atmospheric pressure (about 14.7 psia). In some embodiments, the dehydrogenation reactor 110 may operate at a pressure of about 500 psia. In other embodiments, the dehydrogenation reactor 110 may operate at a pressure from about 4 psia to about 160 psia, from about 20 psia to about 100 psia, or from about 30 psia to about 80 psia. In some embodiments, the processing zone 150 and the regeneration unit 210 may operate with a pressure of within 30 psi of the dehydrogenation reactor 110.

The residence time of the particulate in the dehydrogenation reactor 110 may typically vary from 0.5 seconds (sec) to 240 sec. In other embodiments, the residence time of the particulate solid 180 may be from about 0.5 sec to about 200 sec, from about 0.5 sec to about 100 sec, from about 0.5 sec to about 50 sec, or about 0.5 sec to about 20 sec.

In additional embodiments, the ratio of the particulate solid 180 to the feed stream 102 in the dehydrogenation reactor 110 may range from 5 to 150 on a weight to weight (w/w) basis. In some embodiments, the ratio may range from 10 to 40, such as from 12 to 36, or from 12 to 24.

In additional embodiments, the flux of the particulate solid 180 may be from 1 pound per square foot-second (lb/ft²-s) (about 4.89 kg/m²-s) to 300 lb/ft²-s (to about 97.7 kg/m²-s), such as from 1-20 lb/ft²-s, in the upstream reactor section, and from 1 lb/ft²-s (about 48.9 kg/m²-s) to 300 lb/ft²-s (about 489 kg/m²-s), such as from 10-100 lb/ft²-s, in the downstream reactor section.

In one or more embodiments, the particulate solid 180 may be capable of fluidization. In some embodiments, the particulate solid 180 may exhibit properties known in the industry as “Geldart A” or “Geldart B” properties. Particles may be classified as “Group A” or “Group B” according to D. Geldart, Gas Fluidization Technology, John Wiley & Sons (New York, 1986), 34-37; and D. Geldart, “Types of Gas Fluidization,” Powder Technol. 7 (1973) 285-292, which are incorporated herein by reference in their entireties.

Group A is understood by those skilled in the art as representing an aeratable powder, having a bubble-free range of fluidization; a high bed expansion; a slow and linear deaeration rate; bubble properties that may include a predominance of splitting/recoalescing bubbles, with a maximum bubble size and large wake; high levels of solids mixing and gas backmixing, assuming equal U-Umf (U is the velocity of the carrier gas, and Umf is the minimum fluidization velocity, typically though not necessarily measured in meters per second, m/s, i.e., there is excess gas velocity); axisymmetric slug properties; and no spouting, except in very shallow beds. The properties listed tend to improve as the mean particle size decreases, assuming equal cfp; or as the <45 micrometers (um) proportion is increased; or as pressure, temperature, viscosity, and density of the gas increase. In general, the particles may exhibit a small mean particle size and/or low particle density (<1.4 grams per cubic centimeter, g/cm³), fluidize easily, with smooth fluidization at low gas velocities, and may exhibit controlled bubbling with small bubbles at higher gas velocities.

Group B is understood by those skilled in the art as representing a “sand-like” powder that starts bubbling at Umf; that exhibits moderate bed expansion; a fast deaeration; no limits on bubble size; moderate levels of solids mixing and gas backmixing, assuming equal U-Umf; both axisymmetric and asymmetric slugs; and spouting in only shallow beds. These properties tend to improve as mean particle size decreases, but particle size distribution and, with some uncertainty, pressure, temperature, viscosity, or density of gas seem to do little to improve them. In general, most of the particles having a particle size (cfp) of 40 μm<cfp<500 μm when the density (pp) is 1.4<pp<4 g/cm³, and preferably 60 μm<cfp<500 μm when the density (pp) is 4 g/cm³ and 250 μm<cfp<100 μm when the density (pp) is 1 g/cm³.

Referring still to FIG. 1, in one or more embodiments, the particulate solid 180, and the gas products may be separated within the dehydrogenation reactor 110 by high efficiency cyclones. In embodiments described, the particulate solid 180 may be passed to the processing zone 150 via stream 112. In some embodiments, a portion of the particulate solid 180 may be passed from the processing zone 150 via stream 156 to stream 212 to be used again in the reactor 110 without first passing through the regeneration unit 210. Without being bound by theory it is believed that by recycling a portion of the particulate solid 180 back to the reactor 110 the oxygen-content of the oxygen-carrier material may be better controlled as the oxygen-carrier material may be given more time to react off its oxygen so that the oxygen-carrier material may supply more oxygen to the reactor. It is also believed that recycling the particulate solid 180 may allow for improved temperature control of the reactor 110 because recycling the particulate solid 180 may improve control of the temperature of the materials entering the reactor 110 improving the temperature profile of the reactor 110.

In some embodiments, the dehydrogenation catalyst of the particulate solid 180 may be slightly deactivated after contacting the feed stream 102. In other embodiments, the dehydrogenation catalyst of the particulate solid 180 may still be suitable for reaction in the dehydrogenation reactor 110. As used herein, “deactivated” may refer to a catalyst that is contaminated with a substance such as coke, is cooler in temperature than needed to promote reaction of the feed, or may refer to an oxygen-carrier material that is deficient of oxygen. In some embodiments, a contaminate, such as coke, may be deposited on the particulate solid 180 that is passed from the dehydrogenation reactor 110 to the processing zone 150.

The particulate solid 180 may enter the processing zone 150 via stream 112. The particulate solid 180 may travel in a generally downwards direction through the processing zone 150. A fuel 162 may enter the processing zone 150 through a first gas inlet 160. In some embodiments a stripping gas 172 may enter the processing zone 150 through a second gas inlet 170. The fuel 162 and the optional stripping gas 172 may both travel in a generally upwards direction through the processing zone 150. The particulate solid 180 may exit the processing zone 150 via stream 152 and may be passed to a regeneration unit 210. The gases, such as the fuel 162 and the stripping gas 172 may exit the processing zone 150 via stream 154.

In the regeneration unit 210 the particulate solid 180 may be passed through a gas/solids separator 218. Exiting the separator 218, the particulate solid 180 may pass through an air zone 224 and past the third gas inlet 220 through which the oxygen-containing gas 222 may enter into the regeneration unit 210. The particulate solid 180 may then exit the regeneration unit 210 via stream 212 and be passed back to the dehydrogenation reactor 110.

Regeneration may remove contaminates, such as coke, raise the temperature of the particulate solid 180, increase the oxygen-content of the oxygen-carrier material of the particulate solid 180, or combinations thereof. Regeneration may occur in the processing zone 150, the regeneration unit 210, or both. In some embodiments, the coke on the particulate solid 180 may be removed by combustion with oxygen from the oxygen-carrier material of the particulate solid 180 in the processing zone 150. In some embodiments, any remaining coke on the particulate solid 180 after being passed from the processing zone 150 to the regeneration unit 210 may be removed by combustion in an oxygen-containing environment in the regeneration unit 210. In further embodiments, the particulate solid 180 may be heated by a fuel 162 to a target temperature. The particulate solid 180 may then circulate back to the dehydrogenation reactor 110, carrying the necessary heat for the dehydrogenation reaction. For additional generalized information on dehydrogenations carried out in fluidized beds, those skilled in the art are referred to, for example, US Patent Publication 2005/0177016 A1; International Patent Publication WO 2005/077867 A1 (corresponding to US Patent Publication 2008/0194891 A1); and International Patent Publication WO 20107591 A1.

The oxygen-carrier may be oxidized or reduced as it is utilized in a dehydrogenation process. Different oxidation states of the oxygen-carrier may behave differently within the dehydrogenation process. For example, an oxygen-carrier with a lower oxidation state may not as effectively combust hydrogen within the dehydrogenation reaction as an oxygen-carrier with a comparatively higher oxidation state. Conversely, an oxygen-carrier with a comparatively higher oxidation state may be more likely to oxidize hydrocarbons during the dehydrogenation reaction when compared to an oxygen-carrier with a comparatively lower oxidation state potentially harming the production of olefins from the process. Passing the oxygen-carrier to and through the processing zone 150 and the regeneration unit 210 may help control the oxidation state of the oxygen-carrier material.

In some embodiments, the oxygen-diminished oxygen carrier material of the particulate solid 180 may be re-oxidized to an oxidation state higher than the oxidation state of the oxygen-diminished oxygen carrier material by combustion in an oxygen-containing environment in the regeneration unit 210. In some embodiments, the oxygen-containing environment may be air. In some embodiments of forming the oxygen-rich oxygen carrier material, the oxygen-diminished oxygen carrier material may be restored to its original oxidation state. In some embodiments, the oxygen-diminished oxygen carrier material may have an oxidation state of +2, +3, or +4. The oxygen-rich oxygen carrier material may then circulate back to the dehydrogenation reactor 110, carrying the necessary heat for the dehydrogenation reaction. In other embodiments, nitrogen or steam may also be used to convey the oxygen-rich oxygen carrier material to the dehydrogenation reactor 110. The resulting gas stream 214 from the regeneration unit 210 may consist of air depleted of or containing a lower concentration of O₂.

In one or more embodiments, a supplemental fuel 162 may be combusted in the processing zone 150 to produce heat and increase the temperature of the particulate solid 180. The heat produced by the oxidizing of the oxygen-diminished oxygen carrier material and the combusting of the supplemental fuel 162 may be sufficient to maintain the temperature of the dehydrogenation reactor 110 at a desired temperature. The desired temperature may depend upon the minimum temperature needed for operation of the dehydrogenation reactor 110, since the particulate solid 180 may enter the dehydrogenation reactor 110 and impart its temperature to the dehydrogenation reactor 110.

In one or more embodiments, the processing zone 150 and the regeneration unit 210 may operate at a temperature of 600° C., or even 700° C., to 900° C., such as 725° C. to 875° C., or 750 ° C. to 850° C. Generally, the processing zone 150 and the regeneration unit 210 may have a temperature of at least 50° C. greater than that of the dehydrogenation reactor 110. Such a temperature range may be utilized so that the temperature of the dehydrogenation reactor 110 may be maintained with a limited circulated amount of dehydrogenation catalyst and/or oxygen carrier material. Additionally, such temperatures may be needed to activate the dehydrogenation catalyst if one is utilized.

The residence time of the particulate solid 180 in the processing zone 150 may typically vary from 1 second to 400 seconds. In other embodiments, the residence time of the particulate solid 180 may be from about 1 second to about 350 seconds, from about 1 second to about 300 seconds, from about 1 second to about 250 seconds, from about 1 second to about 200 seconds, from about 1 second to about 150 seconds, from about 1 second to about 100 seconds, from about 1 second to about 50 seconds, or from about 1 second to about 30 seconds.

The residence time of the particulate solid 180 in the regeneration unit 210 may typically vary from 0.5 seconds (sec) to 360 sec. In other embodiments, the residence time of the particulate solid 180 may be from about 0.5 sec to about 200 sec, from about 0.5 sec to about 100 sec, from about 0.5 sec to about 50 sec, or about 0.5 sec to about 20 sec.

The particulate solid 180 and gases from the dehydrogenation reactor 110 may be passed to the processing zone 150 via stream 112. The particulate solid 180 and the gases may be separated from the gases by a gas/solids separator 158. According to one or more embodiments, the separators 158 and 218 may be a riser termination system which may include a cyclonic separation system. In embodiments, where the riser termination system includes a cyclonic separation system some of the particulate solid 180 may be passed out of the riser termination system without first passing through the cyclonic separation system. The cyclonic separation system may include two or more stages of cyclonic separation. In embodiments where the separators 158, 218 comprise more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the particulate solid from the product gas. It should be understood that any primary cyclonic separation device may be used in embodiments of the invention.

In the separator 158 the particulate solid 180 may be separated from product gases and other exhaust gases from the dehydrogenation reactor 110. The gases separated from the particulate solid 180 may combine with gases from the processing zone 150 and exit the processing zone 150 via an exhaust stream 154.

According to embodiments, the particulate solid 180 may move in a generally downward direction through the processing zone 150. As used in the present disclosure the term “generally downward direction” means that the average velocity of the particulate solid 180 is in the downward direction, where the downward direction is with the pull of gravity. As it is an average, the velocity of individual particles of the particulate solid 180 may have a distribution and may not be equal to the average, but taken as a whole the velocity of the particulate solid 180 will average out to be generally downward. According to embodiments, gases within the processing zone 150 may move in a generally upwards direction through the processing zone 150. As used in the present disclosure the term “generally upward direction” means that the average velocity of the gases is in the upward direction, where the upward direction is against the pull of gravity. As it is an average, the velocity of the gas molecules within the regeneration unit 210 may have a distribution and may not be equal to the average, but taken as a whole the velocity of the gases will average out to be generally upward. According to embodiments, the particulate solid 180 and the gases may move in a countercurrent flow pattern through the processing zone 150.

In one or more embodiments, the processing zone 150 may comprise a first gas inlet 160 and a second gas inlet 170. As used herein, “gas inlet” refers to any component operable to inject a gas into the reactor system 100. For example, a gas inlet may be a blower or distributor. Additionally, other suitable gas suppliers are contemplated herein, as would be known by those skilled in the art. In some embodiments, the first gas inlet 160 may be above the second gas inlet 170 as shown in FIG. 1. In some embodiments, the first gas inlet 160 may be below the second gas inlet 170. In further embodiments, the first gas inlet 160 and the second gas inlet 170 may be at the same level within the processing zone 150. In some embodiments, processing zone 150 may only comprise a single gas inlet.

In one or more embodiments, a fuel 162 may enter the processing zone 150 via the first gas inlet 160. In some embodiments, the fuel 162 may comprise hydrogen, methane, ethane, propane, natural gas, or combinations thereof. In some embodiments, the fuel 162 may enter the processing zone 150 in a generally upwards direction. In some embodiments, the concentration of fuel 162 in the processing zone 150 may be from about 5 mol. % to about 100 mol. %, from about 5 mol. % to about 90 mol. %, from about 5 mol. % to about 80 mol. %, from about 5 mol. % to about 70 mol. %, from about 5 mol. % to about 60 mol. %, from about 5 mol. % to about 50 mol. %, from about 5 mol. % to about 40 mol. %, from about 5 mol. % to about 30 mol. %, from about 5 mol. % to about 20 mol. %, from about 5 mol. % to about 10 mol. %, from about 10 mol. % to about 100 mol. %, from about 10 mol. % to about 90 mol. %, from about 10 mol. % to about 80 mol. %, from about 10 mol. % to about 70 mol. %, from about 10 mol. % to about 60 mol. %, from about 10 mol. % to about 50 mol. %, from about 10 mol. % to about 40 mol. %, from about 10 mol. % to about 30 mol. %, from about 10 mol. % to about 20 mol. %, from about 20 mol. % to about 100 mol. %, from about 20 mol. % to about 90 mol. %, from about 20 mol. % to about 80 mol. %, from about 20 mol. % to about 70 mol. %, from about 20 mol. % to about 60 mol. %, from about 20 mol. % to about 50 mol. %, from about 20 mol. % to about 40 mol. %, from about 20 mol. % to about 30 mol. %, from about 30 mol. % to about 100 mol. %, from about 30 mol. % to about 90 mol. %, from about 30 mol. % to about 80 mol. %, from about 30 mol. % to about 70 mol. %, from about 30 mol. % to about 60 mol. %, from about 30 mol. % to about 50 mol. %, from about 30 mol. % to about 40 mol. %, from about 40 mol. % to about 100 mol. %, from about 40 mol. % to about 90 mol. %, from about 40 mol. % to about 80 mol. %, from about 40 mol. % to about 70 mol. %, from about 40 mol. % to about 60 mol. %, from about 40 mol. % to about 50 mol. %, from about 30 mol. % to about 40 mol. %, from about 50 mol. % to about 100 mol. %, from about 50 mol. % to about 90 mol. %, from about 50 mol. % to about 80 mol. %, from about 50 mol. % to about 70 mol. %, from about 60 mol. % to about 100 mol. %, from about 60 mol. % to about 90 mol. %, from about 60 mol. % to about 80 mol. %, from about 60 mol. % to about 70 mol. %, from about 70 mol. % to about 100 mol. %, from about 70 mol. % to about 90 mol. %, from about 70 mol. % to about 80 mol. %, from about 80 mol. % to about 100 mol. %, from about 80 mol. % to about 90 mol. %, or from about 90 mol. % to about 100 mol. %.

In one or more embodiments, at least a portion of the fuel 162 may react with oxygen from the oxygen-carrier material of the particulate solid 180 in the processing zone 150 to produce heat and raise the temperature of at least a portion of the particulate solid 180. Accordingly, in some embodiments, the oxygen content in at least a portion of the oxygen-carrier material of the particulate solid 180 may be reduced in the processing zone 150. In some embodiments, the reaction of the fuel 162 with oxygen from the oxygen-carrier material may produce water. The water may exit the processing zone 150 via stream 154 and be passed to the dehydrogenation reactor 110 where it may combine with the one or more products.

As described herein, in one or more embodiments, coke may be deposited on the particulate solid 180 that may be passed from the dehydrogenation reactor 110 to the processing zone 150. In some embodiments, at least a portion of the coke may be reacted with oxygen from the oxygen-carrier material of the particulate solid 180. However, combustion of coke may be limited, as oxygen-carrier materials may be selective for combusting the fuel 162.

In one or more embodiments, a stripping gas 172 may enter the processing zone 150 via the second gas inlet 170. In some embodiments, the stripping gas 172 may comprise nitrogen, steam, or combinations thereof. In one or more embodiments, the gasses in the processing zone may comprise less than or equal to 1 mol. % O₂. In one or more embodiments, the processing zone 150 may be essentially void of gaseous oxygen. For example, the only source of oxygen in the processing zone 150 may be oxygen from the oxygen-carrier material of the particulate solid 180.

Without being bound by theory, it is believed that by supplying little or no oxygen gas to the processing zone 150 coke that may form on the particulate solid 180 may primarily be burned in the regeneration unit 210 in the presence of the oxygen-containing gas 222. The combustion of coke may produce CO₂ gas which may be difficult to separate from product gases produced in the dehydrogenation reactor 110. Thus it may be advantageous to limit the combustion of coke in the processing zone 150 and to primarily combust coke in the regeneration unit 210 where the CO₂ that may be produced will not significantly mix with the product gases from the dehydrogenation reactor 110 avoiding a difficult separation step. It is believed that without the presence of gaseous oxygen the combustion of coke may be limited.

It also believed that by using a fuel 162 such as hydrogen that does not produce CO₂ as a product of combustion in the processing zone 150 the combustion products can be limited to relatively easier to separate products such as water. Water may be relatively easily separated from the product gases in downstream processes by using a condenser. Thus, by utilizing a processing zone 150 with little to no oxygen gas it is possible to have fuel 162 combustion occur that may primarily produce easily separated combustion products which may allow for the easier processing of the one or more products produced in the dehydrogenation reactor 110.

Additionally, it is believed that the presence of oxygen gas may diminish stripping performance within the processing zone 150, as the oxygen may react with the one or more products, the oxygen-carrier material of the particulate solid 180, or the coke that may form on the particulate solid 180. The primary purpose of using an inert stripping gas 172 may be to limit such reactions so as to exert better control on the conditions within the reactor system 100 and to prevent the carryover of reactive gases, such as oxygen, to other sections of the reactor system 100 as a result the presence of oxygen gas in the processing zone 150 may negatively impact the stripping performance of the processing zone 150.

In one or more embodiments, the processing zone 150 and the dehydrogenation reactor 110 may be contained within a single unit of the reactor system 100. In other embodiments, the processing zone 150 and the dehydrogenation reactor 110 may be separate units of the reactor system 100.

The particulate solid 180 may be passed from the processing zone 150 via stream 152 to a regeneration unit 210. In the regeneration unit 210 the particulate solid 180 may be passed through a gas/solids separator 218. As described herein, in one or more embodiments, the separator 218 may be a riser termination system which may include a cyclonic separation system. In the separator 218 the particulate solid 180 may be separated from exhaust gases such as flue gas and/or carryover gases from other downstream processes. The gases separated from the particulate solid 180 may combine with gases from the regeneration unit 210 and exit the regeneration unit 210 via an exhaust stream 214.

According to embodiments, the particulate solid 180 may move in a generally downward direction through the regeneration unit 210. As used in the present disclosure the term “generally downward direction” means that the average velocity of the particulate solid 180 is in the downward direction, where the downward direction is with the pull of gravity. As it is an average, the velocity of individual particles of the particulate solid 180 may have a distribution and may not be equal to the average, but taken as a whole the velocity of the particulate solid 180 will average out to be generally downward. According to embodiments, gases within the regeneration unit 210 may move in a generally upwards direction through the regeneration unit 210. As used in the present disclosure the term “generally upward direction” means that the average velocity of the gases is in the upward direction, where the upward direction is against the pull of gravity. As it is an average, the velocity of the gas molecules within the regeneration unit 210 may have a distribution and may not be equal to the average, but taken as a whole the velocity of the gases will average out to be generally upward. According to some embodiments, the particulate solid 180 and the gases may move in a countercurrent flow pattern through the regeneration unit 210.

In one or more embodiments, the particulate solid 180 may be exposed to an oxygen-containing gas 222 in the regeneration unit 210. The oxygen-containing gas 222 may enter into the regeneration unit 210 via a third gas inlet 220. In one or more embodiments, the particulate solid 180 may be exposed to the oxygen-containing gas 222 in the regeneration unit 210, such that the content of oxygen in the oxygen-carrier material of the particulate solid 180 may increase in the regeneration unit 210. In some embodiments, the oxygen-containing gas 222 may be air, enriched air, air mixed with steam, or flue gas. In some embodiments, the oxygen-containing gas 222 may include at least 28 mol. % oxygen. In other embodiments, the oxygen-containing gas 222 may include from about 2 mol. % to about 28 mol. % oxygen, from about 2 mol. % to about 25 mol. %, from about 2 mol. % to about 20 mol. %, from about 2 mol. % to about 15 mol. %, from about 2 mol. % to about 10 mol. %, from about 2 mol. % to about 5 mol. %, from about 5 mol. % to about 28 mol. %, from about 5 mol. % to about 25 mol. %, from about 5 mol. % to about 20 mol. %, from about 5 mol. % to about 15 mol. %, from about 5 mol. % to about 10 mol. %, from about 10 mol. % to about 28 mol. %, from about 10 mol. % to about 25 mol. %, from about 10 mol. % to about 20 mol. %, from about 10 mol. % to about 15 mol. %, from about 15 mol. % to about 28 mol. %, from about 15 mol. % to about 25 mol. %, from about 15 mol. % to about 20 mol. %, from about 20 mol. % to about 28 mol. %, from about 20 mol. % to about 25 mol. %, or from about 25 mol. % to about 28 mol. %.

In embodiments, the oxygen-containing gas 222 may enter the regeneration unit 210 in a generally upwards direction. In some embodiments, the area above the third gas inlet 220 may be an air zone 224 as the directional flow of the oxygen-containing gas 222 into the regeneration unit 210 may cause the concentration of oxygen in the area above the third gas inlet 220 to be higher than other areas of the regeneration unit 210. In one or more embodiments, the concentration of oxygen in the air zone 224 may be greater than 25 mol. %. In other embodiments, the concentration of oxygen in the air zone 224 may be from about 4 mol. % to about 28 mol. % oxygen, from about 4 mol. % to about 21 mol. %, from about 4 mol. % to about 10 mol. %, from about 10 mol. % to about 28 mol. %, from about 10 mol. % to about 21 mol. %, or from about 21 mol. % to about 28 mol. % oxygen. The name air zone is utilized here to simply show that oxygen is present since air is often the oxygen-containing gas utilized at least cost.

Now referring to reactor system 200 in FIG. 2, a reactor system according to additional embodiments of the present disclosure is shown. In reactor system 200 the regeneration unit 210 may further comprise a fourth gas inlet 240 beneath the third gas inlet 220. A stripping gas 242 may enter into the regeneration unit 210 through the fourth gas inlet 240. In embodiments, the stripping gas 242 may exit the fourth gas inlet 240 in a generally upwards direction. Accordingly, in embodiments, the area above the fourth gas inlet 240 and below the third gas inlet 220 may be a strip zone 244, as the directional flow of the stripping gas 242 into the regeneration unit 210 may cause the concentration of stripping gas 242 to be higher in the area above the fourth gas inlet 240 and below the third gas inlet 220. In some embodiments, the stripping gas 242 may comprise nitrogen, steam, or combinations thereof.

In one or more embodiments, the stripping gas 242 may comprise a reductant. In one or more embodiments the reductant may comprise hydrogen, methane, or combinations thereof. In some embodiments, the particulate solid 180 may be exposed to the reductant in the stripping gas 242 in the strip zone 244 and the oxygen content in at least a portion of the oxygen-carrier material of the particulate solid 180 may be reduced. In some embodiments, the concentration of reductant in the stripping gas 242 may be greater than 50 mol. %. In some embodiments the concentration of reductant in the stripping gas may be greater than 90 mol. %. In other embodiments the concentration of reductant in the stripping gas 242 may be from about 1 mol. % to about 100 mol. %, such as from about 1 mol. % to about 90 mol. %, from about 1 mol. % to about 80 mol. %, from about 1 mol. % to about 70 mol. %, from about 1 mol. % to about 60 mol. %, from about 1 mol. % to about 50 mol. %, from about 1 mol. % to about 40 mol. %, from about 1 mol. % to about 30 mol. %, from about 1 mol. % to about 20 mol. %, from about 1 mol. % to about 10 mol. %, from about 10 mol. % to about 100 mol. %, from about 10 mol. % to about 90 mol. %, from about 10 mol. % to about 80 mol. %, from about 10 mol. % to about 70 mol. %, from about 10 mol. % to about 60 mol. %, from about 10 mol. % to about 50 mol. %, from about 10 mol. % to about 40 mol. %, from about 10 mol. % to about 30 mol. %, from about 10 mol. % to about 20 mol. %, from about 20 mol. % to about 100 mol. %, from about 20 mol. % to about 90 mol. %, from about 20 mol. % to about 80 mol. %, from about 20 mol. % to about 70 mol. %, from about 20 mol. % to about 60 mol. %, from about 20 mol. % to about 50 mol. %, from about 20 mol. % to about 40 mol. %, from about 20 mol. % to about 30 mol. %, from about 30 mol. % to about 100 mol. %, from about 30 mol. % to about 90 mol. %, from about 30 mol. % to about 80 mol. %, from about 30 mol. % to about 70 mol. %, from about 30 mol. % to about 60 mol. %, from about 30 mol. % to about 50 mol. %, from about 30 mol. % to about 40 mol. %, from about 40 mol. % to about 100 mol. %, from about 40 mol. % to about 90 mol. %, from about 40 mol. % to about 80 mol. %, from about 40 mol. % to about 70 mol. %, from about 40 mol. % to about 60 mol. %, from about 40 mol. % to about 50 mol. %, from about 50 mol. % to about 100 mol. %, from about 50 mol. % to about 90 mol. %, from about 50 mol. % to about 80 mol. %, from about 50 mol. % to about 70 mol. %, from about 50 mol. % to about 60 mol. %, from about 60 mol. % to about 100 mol. %, from about 60 mol. % to about 90 mol. %, from about 60 mol. % to about 80 mol. %, from about 60 mol. % to about 70 mol. %, from about 70 mol. % to about 100 mol. %, from about 70 mol. % to about 90 mol. %, from about 70 mol. % to about 80 mol. %, from about 80 mol. % to about 100 mol. %, from about 80 mol. % to about 90 mol. %, or from about 90 mol. % to about 100 mol. % reductant in the stripping gas 242.

In a first aspect of the present disclosure, hydrocarbons may be dehydrogenated by a method comprising contacting a feed stream comprising one or more hydrocarbons with a particulate solid in a dehydrogenation reactor. The particulate solid comprises an oxygen-carrier material. In the dehydrogenation reactor one or more hydrocarbons are dehydrogenated to form hydrogen and one or more products. At least a portion of the hydrogen is reacted with oxygen from the oxygen-carrier material to form water and reduce the oxygen content in the oxygen-carrier material. The method further comprises passing the particulate solid from the dehydrogenation reactor to a processing zone. A fuel is injected into the processing zone. In the processing zone, at least a portion of the fuel is reacted with oxygen from the oxygen-carrier material of the particulate solid. The gases in the processing zone comprise less than or equal to 1 mol. % O₂. The method further comprises passing at least a portion of the particulate solid from the processing zone to a regeneration unit. The particulate solid is exposed to an oxygen-containing gas in the regeneration unit, such that the content of oxygen in the oxygen-carrier material of the particulate solid is increased in the regeneration unit. The method further comprises passing at least a portion of the particulate solid from the regeneration unit to the dehydrogenation reactor.

A second aspect of the present disclosure may include the first aspect where the method further comprises injecting a stripping gas into the processing zone.

A third aspect of the present disclosure includes any previous aspect or combination of aspects, where the particulate solid moves in a generally downward direction through the processing zone and gases move in a generally upwards direction through the processing zone, such that the particulate solid and gases move in a countercurrent flow pattern through the processing zone.

A fourth aspect of the present disclosure includes any previous aspect or combination of aspects, where the processing zone comprises a first gas inlet and a second gas inlet, a fuel enters into the processing zone through the first gas inlet, and stripping gas enters into the processing zone through the second gas inlet.

A fifth aspect of the present disclosure includes any previous aspect or combination of aspects, where the particulate solid and the oxygen-containing gas in the regeneration unit move generally in a countercurrent flow pattern.

A sixth aspect of the present disclosure includes any previous aspect or combination of aspects, where the reaction of the fuel in the processing zone produces water, and the water is combined with the one or more products.

A seventh aspect of the present disclosure includes any previous aspect or combination of aspects, where coke is deposited on the particulate solid that passes from the dehydrogenation reactor to the processing zone, and at least a portion of the coke is reacted with oxygen from the oxygen-carrier material in the processing zone.

An eight aspect of the present disclosure includes any previous aspect or combination of aspects, where the gases from the processing zone mix with the one or more products.

A ninth aspect of the present disclosure includes any previous aspect or combination of aspects, where the one or more hydrocarbons comprise an alkyl moiety and the one or more products comprise one or more olefinic compounds.

A tenth aspect of the present disclosure includes any previous aspect or combination of aspects, where the fuel is hydrogen.

An eleventh aspect of the present disclosure includes any previous aspect or combination of aspects, where no supplemental fuel is added to the regeneration unit.

A twelfth aspect of the present disclosure includes any previous aspect or combination of aspects, where the particulate solid is exposed to a stripping gas in the regeneration unit and the particulate solid and the stripping gas move generally in a countercurrent flow pattern; and wherein the stripping gas comprises from 0 mol. % to 100 mol. % of a reductant.

A thirteenth aspect of the present disclosure includes any previous aspect or combination of aspects, where the particulate solid consists essentially of the oxygen-carrier material and the dehydrogenation of the one or more hydrocarbons is by non-catalytic thermal dehydrogenation.

A fourteenth aspect of the present disclosure includes any previous aspect or combination of aspects, where the particulate solid further comprises a dehydrogenation catalyst material and the dehydrogenation of the one or more hydrocarbons is at least partially by catalytic dehydrogenation.

A fifteenth aspect of the present disclosure includes any previous aspect or combination of aspects, where the dehydrogenation catalyst material and the oxygen-carrier material are separate particles of the particulate solid or the dehydrogenation catalyst material and the oxygen-carrier material are contained in the same particles of the particulate solid.

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

For the purposes of describing and defining the present disclosure it is noted that the term “about” are utilized in this disclosure to represent the inherent degree of uncertainty that may be attributed to any quantitative comparison, value, measurement, or other representation. The term “about” are also utilized in this disclosure to represent the degree by which a quantitative representation may vary from a stated reference without resulting in a change in the basic function of the subject matter at issue.

In relevant cases, where a composition is described as “comprising” one or more elements, embodiments of that composition “consisting of” or “consisting essentially of” those one or more elements is contemplated herein.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

In some embodiments, chemicals or chemical streams are described as “passing” from one system unit or portion of a system unit to another. As described herein, such passing may include direct passing or indirect passing. For example, when passing from “unit A” to “unit B”, direct passing has no intermediate destination between unit A and unit B (i.e., directly through a pipe or other transport passage), and indirect passing may include one or more intermediate destinations between unit A and unit B. For example, a stream passing from unit A to unit B may passed through, without limitation, a heat exchanger, treatment device, etc.

It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein.

1. A method for dehydrogenating hydrocarbons, the method comprising:
contacting a feed stream comprising one or more hydrocarbons with a particulate solid in a dehydrogenation reactor, wherein the particulate solid comprises an oxygen-carrier material, and wherein in the dehydrogenation reactor:
the one or more hydrocarbons are dehydrogenated to form hydrogen and one or more products; and
at least a portion of the hydrogen is reacted with oxygen from the oxygen-carrier material to form water and reduce the oxygen content in the oxygen-carrier material;
passing the particulate solid from the dehydrogenation reactor to a processing zone, wherein:
fuel is injected into the processing zone;
in the processing zone, at least a portion of the fuel is reacted with oxygen from the oxygen-carrier material of the particulate solid;
the gasses in the processing zone comprise less than or equal to 1 mol. % O₂;
passing at least a portion of the particulate solid from the processing zone to a regeneration unit, wherein the particulate solid is exposed to an oxygen-containing gas in the regeneration unit such that the content of oxygen in the oxygen-carrier material of the particulate solid is increased in the regeneration unit; and
passing at least a portion of the particulate solid from the regeneration unit to the dehydrogenation reactor.

2. The method of claim 1, further comprising injecting a stripping gas into the processing zone.

3. The method of claim 1, wherein the particulate solid moves in a generally downward direction through the processing zone and gases move in a generally upwards direction through the processing zone, such that the particulate solid and gases move in a countercurrent flow pattern through the processing zone.

4. The method of claim 1, wherein the processing zone comprises a first gas inlet and a second gas inlet, a fuel enters into the processing zone through the first gas inlet, and stripping gas enters into the processing zone through the second gas inlet.

5. The method of claim 1, wherein the particulate solid and the oxygen-containing gas in the regeneration unit move generally in a countercurrent flow pattern.

6. The method of claim 1, wherein the reaction of the fuel in the processing zone produces water, and the water is combined with the one or more products.

7. The method of claim 1, wherein coke is deposited on the particulate solid that passes from the dehydrogenation reactor to the processing zone, and at least a portion of the coke is reacted with oxygen from the oxygen-carrier material in the processing zone.

8. The method of claim 1, wherein the gases from the processing zone mix with the one or more products.

9. The method of claim 1, wherein the one or more hydrocarbons comprise an alkyl moiety and the one or more products comprise one or more olefinic compounds.

10. The method of claim 1, wherein the fuel comprises hydrogen.

11. The method of claim 1, wherein no supplemental fuel is added to the regeneration unit.

12. The method of claim 1, wherein the particulate solid is exposed to a stripping gas in the regeneration unit and the particulate solid and the stripping gas move generally in a countercurrent flow pattern; and wherein the stripping gas comprises from 0 mol. % to 100 mol. % of a reductant.

13. The method of claim 1, wherein the particulate solid consists essentially of the oxygen-carrier material and the dehydrogenation of the one or more hydrocarbons is by non-catalytic thermal dehydrogenation.

14. The method of claim 1, wherein the particulate solid further comprises a dehydrogenation catalyst material and the dehydrogenation of the one or more hydrocarbons is at least partially by catalytic dehydrogenation.

15. The method of claim 14, wherein the dehydrogenation catalyst material and the oxygen-carrier material are separate particles of the particulate solid or the dehydrogenation catalyst material and the oxygen-carrier material are contained in the same particles of the particulate solid.
